(19) United States
(12) Patent Application Publication
(10) Pub. No.: US 2007/0167898 A1
(43) Pub. Date: Jul. 19, 2007

(54) Methods and devices for tensioning a wrap around a blood vessel

(75) Inventors: William Suttle Peters, Auckland (NZ); Scott Hugh Miller, Manly (AU); Gemma Au-Yeung, Waitara (AU)

Correspondence Address: ORRICK, HERRINGTON & SUTCLIFFE, LLP;IP PROSECUTION DEPARTMENT, 4 PARK PLAZA, SUITE 1600, IRVINE, CA, 92614-2558, US

(73) Assignee: SUNSHINE HEART COMPANY PTY LTD.

(21) Appl. No.: 10/595,604

(22) PCT Filed: Oct. 28, 2004
(86) PCT No.: PCT/AU04/01488

§ 371 Date: Mar. 21, 2007

(30) Foreign Application Priority Data
Oct. 30, 2003 (AU) 2003905995

(51) Int. Cl. A61L 15/00

(52) U.S. Cl. 602/75

(57) ABSTRACT

A method of securing a flexible wrap (30) around a blood vessel (28). The wrap (30) being generally elongate and having first (30a) and second (30b) end portions. The method including the steps of 1. wrapping the flexible wrap around the blood vessel (28); 2. passing the first end (30a) of the wrap (30) through a buckle device (10) affixed substantially distally from the first end (30a) of the wrap (30); 3. adjusting the tension in the wrap (30) to a desired level by movement of the first end (30a) of the wrap (30) relative to the buckle device (10); 4. securing together adjacent parts of the wrap (30) substantially adjacent the blood vessel (28); and 5. removing the buckle device (10).

### TECHNICAL FIELD

The present invention relates to a method of tensioning a wrap around a blood vessel, such as an arterial vessel, and an associated tensioning device.

The invention has been primarily developed for use in securing an inflatable balloon or chamber of an implantable counter-pulsation heart assist device against the ascending aorta and will be described hereinafter with reference to this application. However, the invention also finds broader application in the tensioning of wraps around any blood vessel, whether static or dynamic, applied to either an artery or vein.

### BACKGROUND OF THE INVENTION

The Applicant's International PCT Patent Application Nos. PCT/AU00/00654 and PCT/AU01/01187 disclose heart assist devices, systems and methods. More particularly, these specifications disclose vessel deformers in the form of inflatable balloon or chambers which form part of implantable counter-pulsation heart assist devices. The balloon or chambers are cyclically inflated and deflated and used to compress the patient's ascending aorta during diastole and release the compression during systole.

The balloon or chamber are generally secured to the aorta by a wrap or sheath, which is secured around a section of the aorta with the balloon or chamber between the wrap and the vessel. For the heart assist device to function efficiently, it is necessary that the wrap be a snug fit around the aorta when the balloon or chamber is deflated.

During the implantation of known heart assist devices, the wrap is pulled tight around the aorta and held by forceps or similar clamps whilst the regions of the wrap adjacent to the aorta are sutured together. It is difficult for a surgeon to judge exactly how tight the wrap is during this procedure. It is also difficult for repeatable tension to be applied to wraps or for the wrap to be conformal about the length of the wrap (i.e. for the tension to be evenly spread along the length of the wrap).

It is also known to apply static wraps to the exterior of blood vessels, for instance to strengthen a vessel suffering from aneurysmal disease. It is also similarly difficult to appropriately adjust the tension of such static wraps when they are applied to the vessel to be reinforced.

It is an object of the present invention to substantially overcome or at least ameliorate one or more of the above disadvantages.

### SUMMARY OF THE INVENTION

Accordingly, in a first aspect, the present invention provides a method of securing a flexible wrap around a blood vessel, the wrap being generally elongate and having first and second end portions, the method including the steps of:

(1) wrapping the flexible wrap around the blood vessel;

(2) passing the first end portion of the wrap through a buckle device affixed substantially distally from the first end portion of the wrap;

(3) adjusting the tension in the wrap to a desired level by movement of the first end portion of the wrap relative to the buckle device;

(4) securing together adjacent parts of the wrap substantially adjacent the blood vessel; and

(5) removing the buckle device.

The buckle device preferably includes means to hold the adjacent parts of the wrap together.

The wrap preferably holds a heart assist device vessel deformer in place, most preferably against an arterial vessel.

Step 4 preferably involves securing by suturing or stapling—sutures may be or known materials such as non-absorbable sutures e.g. Prolene™ or silk, or nitinol sutures or staples, or absorbable sutures such as cat-gut or Vicryl™.

The method preferably also includes the step of trimming off the parts of the wrap external to the sutures.

The method preferably also includes the step of releasably attaching the buckle device to the wrap prior to Step 1. In one form, the buckle device is sutured to the second end portion of the wrap. In another form, the buckle device includes a leg or legs that respectively pierces or pierce the wrap. In another form, the buckle device includes a pair of spring legs that are adapted to clamp the wrap therebetween. These latter buckle systems are “self-holding”.

In one embodiment, the buckle device preferably includes at least two parallel and spaced apart legs and the method preferably includes adjusting the tension in the wrap until the legs begin to deform towards each other.

In another embodiment, the wrap includes aortic circumference distance markers, and the method preferably includes adjusting the tension in the wrap until the desired aortic circumference is reached.

In still another embodiment, the buckle is adapted to lightly grip the first and second end portions of the wrap so that the wrap may be drawn tight around the vessel and then released. In this embodiment of the invention the buckle may be attached the second end portion of the wrap. More preferably, the buckle is adapted to allow the wrap to begin to pull through the buckle when the tension applied to the wrap to the vessel just equals the holding force of the buckle on the wrap—this allows a relatively repeatable tension to be applied to the wrap.

The present invention further consists in a flexible wrap adapted to be secured around a blood vessel within a patient, the wrap being generally elongate and having first and second end portions, there being attached to the wrap a buckle device through which the second end portion of the wrap may be threaded to allow the wrap to be drawn to a desired tension about the blood vessel, the buckle device being removable from the wrap after the end portions thereof have been connected together around the blood vessel.

The buckle device preferably includes means to hold overlapping parts of the wrap together.

The buckle device is preferably attached to the wrap substantially distally to the second end portion. The buckle device is preferably attached to the wrap adjacent to the first end portion.

The end portions are preferably sutured together.

In another aspect, the present invention provides a heart assist device wrap for use in securing a vessel deformer to an arterial vessel, the wrap being generally elongate with two end portions and having a buckle device releasably attached thereto that includes at least a pair of substantially parallel legs with a gap therebetween through which the two end portions of the wrap can pass.

In a further aspect, the present invention provides a buckle device for use in securing a wrap around an arterial vessel, the wrap being generally elongate and having two end portions, the buckle device including at least a pair of substantially parallel legs with a gap therebetween through which the two end portions of the wrap can pass, wherein at least one of the legs is adapted for releasably fixing to the wrap.

In one form, the buckle device is adapted for suturing to the wrap. In this form the buckle has loops through which the buckle device is attached to one end of the wrap so that the other end of the wrap can be pulled without the buckle moving relative to the first end.

In another form, the buckle device is adapted for stapling to the wrap.

In a further form, the device includes a pair of enlarged ends adapted to clear suture knots during removal of the device from the secured wrap. The device may also include an enlarged formation in about the middle of one the legs, which is adapted to allow forcep access between the two legs to facilitate grasping of the second end of the wrap to draw the wrap end through the buckle. The other leg of the device is preferably formed from two part legs stemming from each of the enlarged end formations, the two part legs having a small clearance between their distal ends. The clearance is adapted to facilitate removal of the buckle from any sutures that are used to secure the wrap yet are still continuous to the wrap at the time of buckle removal.

It is desirable to have the buckle device hold itself in position whilst the wrap is secured to itself, as opposed to requiring the use of surgical clamps or the like. The is buckle device may include a third leg that pierces the wrap. In this form, the three legs of the buckle device are all substantially parallel, with the first and second legs being joined at one end of the wrap and the other end of the wrap is passed between the second and third legs and adjusted to the desired wrap tension. The second and third legs act to compress and hold the wrap in position. Alternatively, small barbs can be placed on one of the legs, such that as the wrap end is pulled through, the material runs forward over the barb, and on pulling back, the barbs snag into the wrap to secure it in position whilst the wrap is secured.

A further form of the invention utilises spring wire and telltales to indicate the tension developed when pulling on the wrap to secure it around the blood vessel. The arms of the buckle are formed and sized relative to spring force such that when the wrap is appropriately tensioned the arms deflect towards one another. A further feature can be added to indicate the degree of tension by the use of over-lapping perpendicular arms to the flexure.

In another approach, the buckle is adapted to prevent accidental removal during its use. In one form, loops in one side of the parallel legs are provided to secure the buckle to the wrap. The loops provide secure attachment of the buckle by preventing migration of the buckle from its attaching sutures. The loops can be replaced by bends such a V or U, alternatively a tubular shape can be fit and secured to the legs to perform the same function.

In a further alternative the buckle is curved to replicate the adjacent curve of a blood vessel, such as an aorta.

### BRIEF DESCRIPTION OF THE DRAWINGS

Preferred embodiments the invention will now be described, by way of examples only, with reference to the accompanying drawings in which:

FIG. 1 is a perspective view of a first embodiment of a buckle device according to the invention;

FIGS. 2 to 4 sequentially show a heart assist device being secured to an arterial vessel using the buckle device shown in FIG. 1;

FIG. 5 is a plan view of the second embodiment of buckle device according to the invention;

FIGS. 6 to 9 sequentially show a heart assist device being secured to an aorta using the buckle device shown in FIG. 5;

FIG. 10 is a perspective view of a third embodiment of a buckle device according is to the invention;

FIG. 11 show a wrap secured to around an invisible arterial vessel using the buckle device shown in FIG. 10; and

FIGS. 12 to 15 are plan views of fourth to seventh embodiments of buckle devices according to the invention.

### DETAILED DESCRIPTION OF THE PREFERRED EMBODIMENT

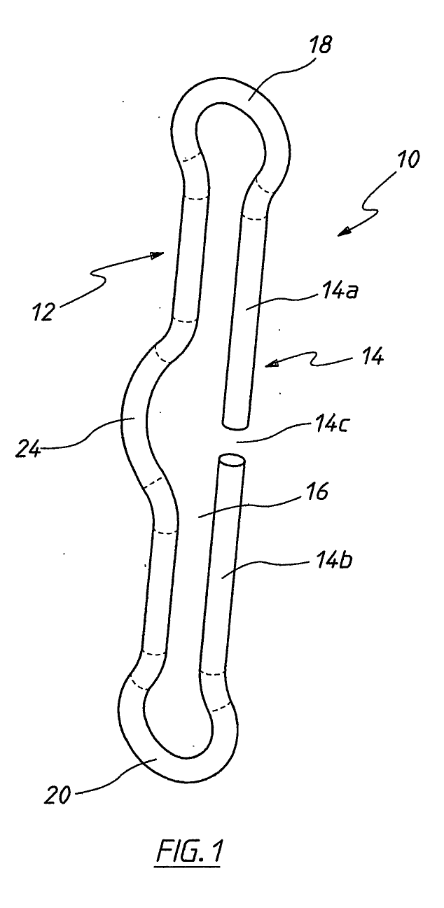

FIG. 1 shows a first embodiment of a buckle device 10 according to the invention. The device 10 is formed from stainless steel wire of approximately 1 mm in diameter. Depending on application, the wire diameter can vary from 0.1-1.5 mm, and could alternatively be molded from plastic.

The device 10 includes a first leg 12 and a second leg 14. The legs 12, 14 are substantially parallel and spaced apart by a gap 16. The device 10 has bulbous ends 18 and 20 and a bulbous formation 24 in the middle of the leg 12, the purposes of which will be described below. The second leg 14 is formed from two leg parts 14a and 14b which have a small clearance 14c between their distal ends, the purpose of which will also be described in more detail below.

FIG. 2 shows a heart assist device 26 being secured to a section of aorta 28 (shown in phantom lines) by a substantially inelastic flexible wrap 30, which is preferably made from polyester or similar plastics material. The wrap 30 may be made from a sheet of woven material such as that sold under the trade mark Dacron or may be made from a sheet of a film like material such as that sold under the trade mark GoreTex. The wrap 30 is longitudinal in shape and has first and second ends 30a, 30b.

Prior to the operation to implant the heart assist device 26, the buckle device 10 is attached to the wrap 30, near the end 30a, by sutures 32. Put another way, the buckle device 10 is attached to the wrap 30 remote or distal from the end 30b.

The heart assist device 26 is then positioned on the exterior of the aorta 28 and the wrap 30 is placed over the heart assist device 26 and around the aorta 28. The end 30b of the wrap 30 is then pulled through the gap 16 into the position shown in FIG. 2. The bulbous formation 24 in the leg 14, 12 provides convenient access for forceps to reach between the two legs 14, 16 and grasp the end 30b of the wrap 30 to pull it through the gap 16.

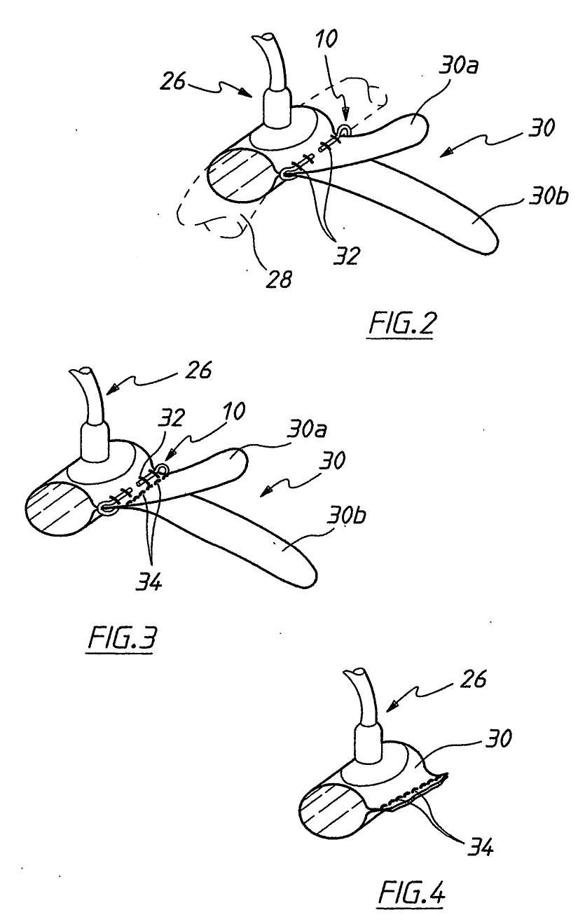

The surgeon then grasps the two ends 30a, 30b of the wrap 30 and pulls them in substantially opposite directions until the legs 12, 14 of the buckle device 10 begin to resiliently deform. This initial deformation provides the surgeon with a repeatable indication of the preferred level of tension in the wrap 30. The ends 30a, 30b of the wrap 30 are then maintained at this preferred position and tension whilst they are joined together with sutures 34, as shown in FIG. 3.

To complete the implantation, the parts of the wrap 30 external to the sutures 34 are cut off, as shown in FIG. 4. The sutures 32 securing the buckle device 10 to the wrap 30 are then cut so the buckle device 10 can be slid over the sutures 34 and removed. The bulbous ends 18, 20 of the buckle device 10 provide clearance for any knots of the sutures 34 that may be encountered during the removal of the buckle device 10. The clearance 14c also facilitates removal of the buckle 10 from any sutures that are used to secure the wrap 30 yet are still continuous to the wrap 30 at the time of buckle removal.

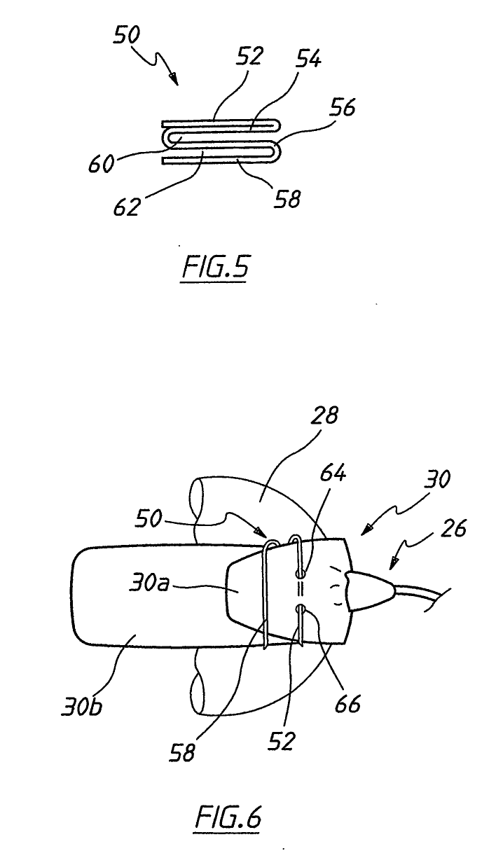

FIG. 5 shows a second embodiment of the buckle device 50 according to the invention. The device 50 will now be described with reference to FIGS. 6 to 9 and like reference numerals to those shown in relation to the first embodiment will be used to indicate like features in the second embodiment.

The buckle device 50 is also made from stainless steel wire and is formed from four legs 52, 54, 56 and 58. The two legs 52 and 54 are folded back closely against one another so that they grip the wrap 30 when it is forced therebetween. The legs 54, 56 and 58 are all equally spaced apart with gaps 60 and 62 therebetween.

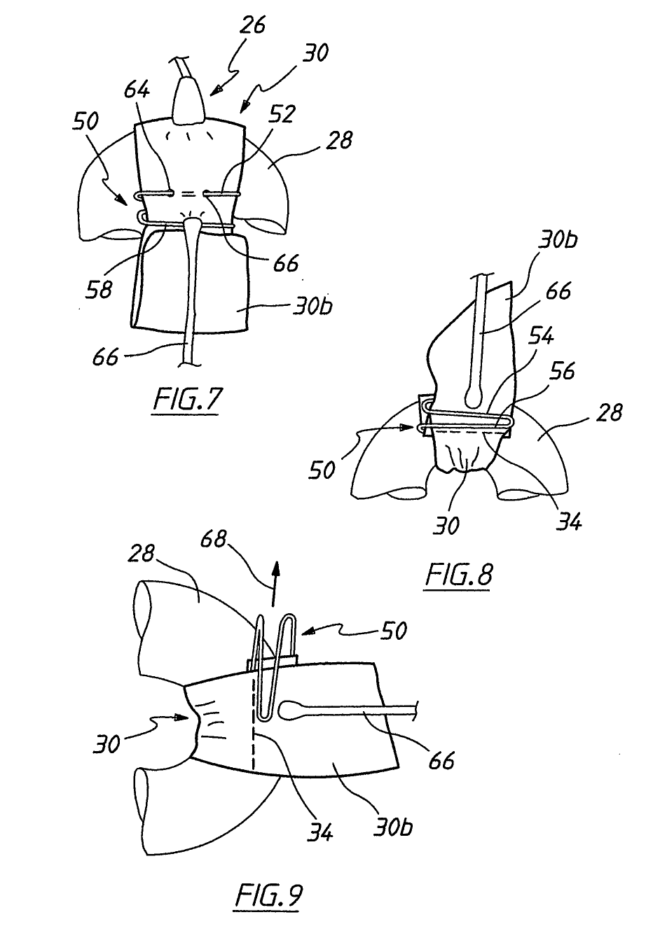

The buckle device 50 is attached to end 30a of the wrap 30 prior to commencement of the surgical procedure. This attachment is achieved by inserting the leg 52 through two holes 64 and 66 in the wrap 30, as shown in FIG. 6. Threading the leg 52 through the wrap 30 in this way, in combination with the wrap also being clamped between the two legs 52, 54, ensures a secure attachment. The wrap 30 is then positioned around the aorta as shown in FIG. 6 with the other end 30b threaded through the gaps 60 and 62. Forceps 66 are then used to pull the other end 30b of the wrap 30 through the gaps 60, 62, as shown in FIG. 7.

As is shown in FIG. 8, the forceps 66 are then used to move the end 30b of the wrap 30 relative to the buckle device 50 in order to tension same. The wrap 30 is tensioned until the leg 58 begins to resiliently deform, which again provides a repeatable indication of wrap tension to the surgeon. The two ends 30a, 30b of the wrap 30 are then sutured together by sutures 34. When the suturing has been completed, the buckle device 50 is removed by sliding it away from the wrap 30 in the direction of arrow 68. The parts of the wrap 30 external the sutures 34 can then be trimmed off.

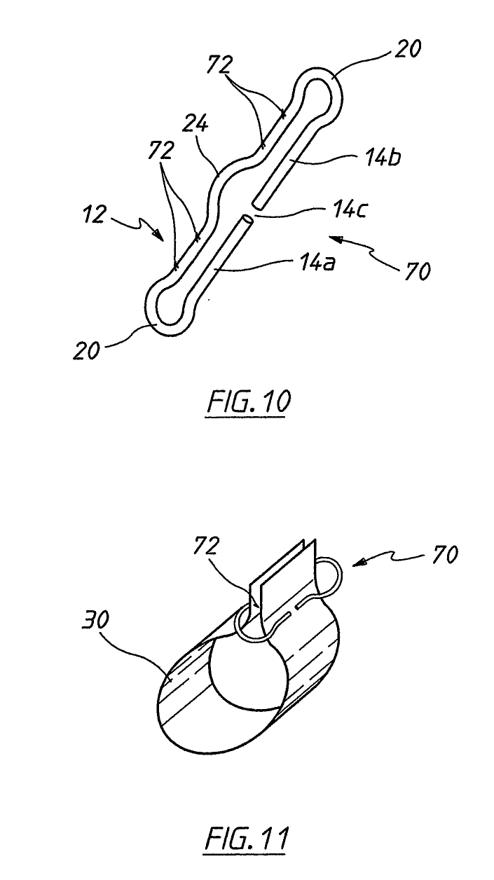

FIGS. 10 and 11 show a third embodiment of buckle device 70 according to the invention. Like reference numerals to those used in relation to the first embodiment will be used to indicate like features in the third embodiment. The device 70 is similar to the first embodiment except it also includes small angled hooks or barbs 72, which provide a self holding or non return function to maintain the ends of the wrap 30a, 30b in their preferred position during their suturing together.

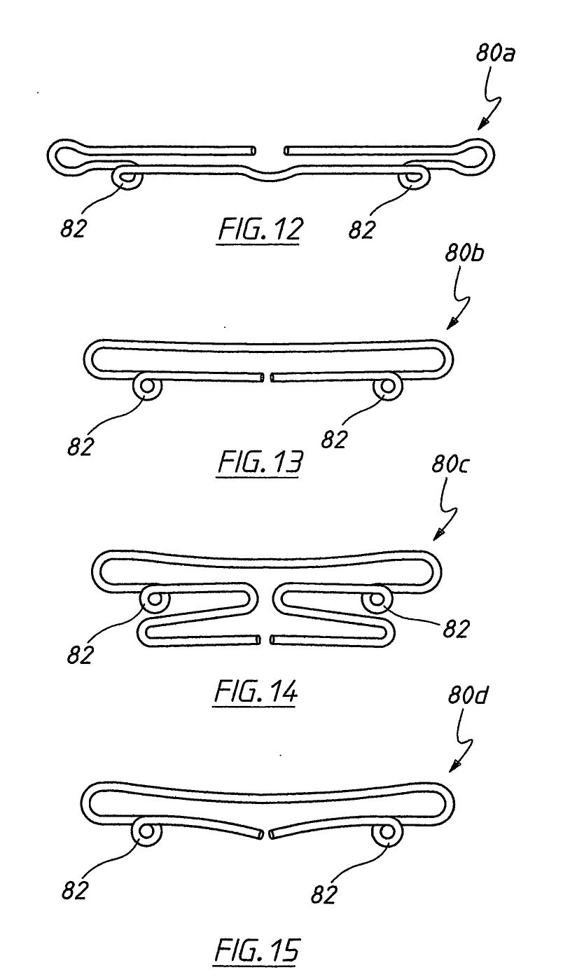

FIGS. 12 to 15 respectively show fourth to seventh embodiments of buckle device 80a, 80b, 80c, and 80d, according to the invention. Like reference numerals to those used in relation to the first embodiment will be used to indicate like features in these embodiments. The devices 80a, 80b, 80c, and 80d all include looped portions 82 through which tacking sutures may be threaded to hold the buckle in place on the wrap.

The main advantage of the devices and methods disclosed above is that they provide a consistent and repeatable indication of wrap tension to the surgeon, which enables the ends of the wrap to be accurately positioned prior to their connection by suturing.

Another advantage is that the buckle device acts as a guide to suturing of the wrap to itself. The suture needle can be skimmed just above the buckle and thus the buckle acts to reduce the risk of the suture needle or staple etc to inadvertently puncture the underlying inflatable balloon.

It will be appreciated by persons skilled in the art that numerous variations and/or modifications can be made to the invention as shown in the specific embodiments without departing from the spirit or scope of the invention as broadly defined.

For example, the buckle devices could alternatively be made of plastic. Additionally, absorbable suture material may be used if the heart assist device is only required for a finite period (eg. two to three weeks or up to 6 months, depending on the suture material used), which would also then allow percutaneous removal. If desired the wrap may be attached to the heart assist device before placement into the patient's body.

1. A method of securing a flexible wrap around a blood vessel, the wrap being generally elongate and having first and second end portions, the method including the steps (1) wrapping the flexible wrap around the blood vessel; (2) passing the first end portion of the wrap through a buckle device affixed substantially distally from the first end portion of the wrap; (3) adjusting the tension in the wrap to a desired level by movement of the first end portion of the wrap relative to the buckle device; (4) securing together adjacent parts of the wrap substantially adjacent the blood vessel; and (5) removing the buckle device.

2. The method as claimed in claim 1, wherein the buckle device includes means to hold the adjacent parts of the wrap together.

3. The method as claimed in claim 1, wherein the wrap holds a heart assist device vessel in place.

4. The method as claimed in claim 3, wherein the wrap holds a heart assist device vessel in place against an arterial vessel.

5. The method as claimed in claim 1, wherein Step 4 involves securing by suturing.

6. The method as claimed in claim 1, wherein Step 4 involves securing by stapling.

7. The method as claimed in claim 1, wherein the method also includes the step of trimming off the parts of the wrap external to the sutures.

8. The method as claimed in claim 1, wherein the method also includes the step of releasably attaching the buckle device to the wrap prior to Step 1.

9. The method as claimed in claim 8, wherein the buckle device is sutured to the second end portion of the wrap.

10. The method as claimed in claim 8, wherein the buckle device is stapled to the second end portion of the wrap.

11. The method as claimed in claim 8, wherein the buckle device includes a leg or legs that respectively pierces or pierce the wrap.

12. The method as claimed in claim 6, wherein the buckle device includes a pair of spring legs that are adapted to clamp the wrap therebetween.

13. The method as claimed in claim 1, wherein the buckle device includes at least two parallel and spaced apart legs and the method includes adjusting the tension in the wrap until the legs begin to deform towards each other.

14. The method as claimed in claim 1, wherein the wrap includes aortic circumference distance markers, and the method includes adjusting the tension in the wrap until the desired aortic circumference is reached.

15. The method as claimed in claim 1, wherein the buckle device is adapted to lightly grip the first and second end portions of the wrap so that the wrap may be drawn tight around the vessel and then released.

16. A flexible wrap adapted to be secured around a blood vessel within a patient, the wrap being generally elongate and having first and second end portions, there being attached to the wrap a buckle device through which the second end portion of the wrap may be threaded to allow the wrap to be drawn to a desired tension about the blood vessel, the buckle device being removable from the wrap after the end portions thereof have been connected together around the blood vessel.

17. The wrap as claimed in claim 16, wherein the buckle device includes means to hold overlapping parts of the wrap together.

18. The wrap as claimed in claim 16, wherein the buckle device is attached to the wrap substantially distally to the second end portion.

19. The wrap as claimed in claim 18, wherein the buckle device is attached to the wrap adjacent to the first end portion.

20. The wrap as claimed in claim 16, wherein the end portions are sutured together.

21. A heart assist device wrap for use in securing a vessel to an arterial vessel, the wrap being generally elongate with two end portions and having a buckle device releasably attached thereto that includes at least a pair of substantially parallel legs with a gap therebetween through which the two end portions of the wrap can pass.

22. A buckle device for use in securing a wrap around an arterial vessel, the wrap being generally elongate and having two end portions, the buckle device including at least a pair of substantially parallel legs with a gap therebetween through which the two end portions of the wrap can pass, wherein at least one the legs is adapted for releasably fixing the wrap.

23. The device as claimed in claim 22, wherein the buckle device is adapted for suturing to the wrap.

24. The device as claimed in claim 22, wherein the buckle device is adapted for stapling to the wrap.

25. The device as claimed in claim 23, wherein the device includes a pair of enlarged ends adapted to clear suture knots during removal of the device from the secured wrap.

26. The device as claimed in claim 25, wherein the device also includes an enlarged formation in about the middle of one the legs, which is adapted to allow forceps access between the two legs.

27. The device as claimed in claim 26, wherein the other leg of the device is formed from two part legs stemming from each of the enlarged end formations, the two part legs having a small clearance between their distal ends.

28. The device as claimed in claim 23, wherein the buckle device includes a third leg that pierces the wrap.

29. The device as claimed in claim 28, wherein the three legs of the buckle device are all substantially parallel, with the first and second legs being joined at one end of the wrap and the other end of the wrap is passed between the second and third legs and adjusted to the desired wrap tension.

30. The device as claimed in claim 23, wherein small barbs are included on one of the legs, such that as the wrap end is pulled through, the material runs forward over the barb, and on pulling back, the barbs snag into the wrap to secure it in position whilst the wrap is secured.

31. The device as claimed in claim 23, wherein the buckle device utilizes spring wire and telltales to indicate the tension developed when pulling on the wrap to secure it around the blood vessel.

32. The device as claimed in claim 31, wherein the arms of the buckle are formed and sized relative to spring force such that when the wrap is tensioned the arms deflect towards one another.

33. The device as claimed in claim 23, wherein loops in one side of the parallel legs are provided to secure the buckle to the wrap.

34. The device as claimed in claim 23, wherein bends or a tubular shape in one side of the parallel legs are provided to secure the buckle device to the wrap.

35. The device as claimed in claim 23, wherein the buckle is curved to replicate the adjacent curve of a blood vessel.
